(19) United States
(12) Patent Application Publication
(10) Pub. No.: US 2007/0207126 A1
(43) Pub. Date: Sep. 6, 2007

(54) Therapeutic microencapsulation of embedding parathyroid live cells with theracyte and a manufacturing method thereof

(75) Inventor: Fong-Fu Chou, Gueishan Township (TW)

Correspondence Address: NIKOLAI & MERSEREAU, P.A., 900 SECOND AVENUE SOUTH, SUITE 820, MINNEAPOLIS, MN, 55402, US

(73) Assignee: Non-Profit Organization Chang Gung Memorial Hospital, Gueishan Township (TW)

(21) Appl. No.: 11/366,231

(22) Filed: Mar. 2, 2006

(51) Int. Cl. A61K 35/55

(52) U.S. Cl. 424/93.7

(57) ABSTRACT

A therapeutic microencapsulation for embedding parathyroid live cells with TheraCyte® and a manufacturing method are disclosed, where in the agent is made by embedding at least 4×10⁵ parathyroid live cells with TheraCyte®. The achieved agent can be planted into mammal objects for treating osteoporosis in long-term. The manufacturing method is characterized by immerging the parathyroid live gland in composition of Roswell Park Memorial Institute solution (85%), dimethyl sulfoxide (10%) and fetal calf serum 5% and depositing in −179° C. liquid nitrogen. The frozen parathyroid gland is sliced to pieces to isolate the live cells after unfreezing and then the parathyroid live cells are embedded with TheraCyte® to achieve the therapeutic microencapsulation.

### BACKGROUND OF THE INVENTION

1. Field of the Invention

The present invention relates to a therapeutic microencapsulation of embedding parathyroid live cells and a manufacturing method thereof, and more particularly to a therapeutic microencapsulation contains 4×10⁵ or 4×10⁶ parathyroid live cells in TheraCyte® to treat diseases such as osteoporosis and hypoparathyroidism.

2. Description of Related Art

In recent years, parathyroid hormone, more particularly the synthesized parathyroid hormone by gene engineering, is subjected to cure osteoporosis. However, the parathyroid hormone is composed of amino acid, the patent needs one dosage a day by subcutaneous injection.

A currently developed parathyroid hormone named in “teriparatide” (Forteo) contains 3 c.c., 750 mg in a tube for one month use, 25 mg subcutaneous injection a day. Although the patent does not need to change medicines everyday for convenience purposes, the patent has to keep on injecting the parathyroid hormone. Once the patent stops taking the parathyroid hormone, the bone mineral loses quickly. In therapeutics, continuously daily injection is troublesome but necessary.

### SUMMARY OF THE INVENTION

A first main objective of the present invention is to provide a therapeutic microencapsulation that contains parathyroid live cells and is implanted inside mammal objects for about 4 months active duration so that daily parathyroid hormone injection is avoided.

A second main objective of the present invention is to provide a manufacturing method that produces the therapeutic microencapsulation.

To achieve the first objective, the therapeutic microencapsulation is made by embedding at least 4×10⁵ parathyroid live cells in TheraCyte®, wherein the parathyroid live cells are contained in −179° C. liquid nitrogen and then unfrozen for preparing the therapeutic microencapsulation. By implanting the therapeutic microencapsulation into a mammal object, the parathyroid live cells last four months to increase bone mineral density of the object and to efficiently treat osteoporosis.

To achieve the second main objective, the manufacturing method comprises steps of:

obtaining a parathyroid gland, wherein the parathyroid gland is obtained by separating a heaviest parathyroid gland in surgery from a mammal having repeating hyperparathyroidism;

refrigerating the parathyroid gland, wherein the parathyroid gland is immerged by a composition composed of Roswell Park Memorial Institute solution (85%), dimethyl sulfoxide (10%) and fetal calf serum (5%) and contained in −179° C. liquid nitrogen;

isolating the parathyroid cells, wherein the refrigerated parathyroid gland is unfrozen and sliced into piece to isolate the parathyroid cells, microspheres are suspended in a full growth solution and then a trypsin/EDTA 0.05 solution is used to individualize the parathyroid cells; and

embedding to achieve the therapeutic microencapsulation, wherein at least 4×10⁵ parathyroid cells are embedded in Theracyte® to achieve the therapeutic microencapsulation.

In characteristics, the sliced parathyroid gland after unfreezing further is treated to isolate parathyroid cells by using Collagenase II.

Wherein, the amount of the parathyroid live cells is calculated by an excluding method of trypan blue after separating the parathyroid cells with the trypsin/EDTA 0.05% solution.

By embedding at least 4×10⁵ parathyroid live cells in Theracyte® and preserving the parathyroid cells in −179° C. liquid nitrogen before embedding, the therapeutic microencapsulation can achieve a four-month medicinal duration in implantation to a mammal object to substitute daily injection in the conventional usage.

Further benefits and advantages of the present invention will become apparent after a careful reading of the detailed description with appropriate reference to the accompanying drawings.

### BRIEF DESCRIPTION OF THE DRAWINGS

FIG. 1 is a chart showing bone density variations at lumbar spines (L1-L5) and left femurs of white mice after ovariectomy, one-month period and three-month period, wherein the bone density is decreased in the lumbar spines and meaninglessly increased in the left femurs in a control group; the bone density is meaningfully increased in the lumbar spines and the left femurs in the TheraCyte® A (A-group) and TheraCyte® B groups (B-group);

FIG. 2 is a picture showing cells alive that are tested by H & E staining after embedding 4×10⁶ parathyroid live cells into TheraCyte® one month later;

FIG. 3 is a picture showing parathyroid cells alive that are tested by immune tissue staining after embedding 4×10⁶ parathyroid live cells into TheraCyte® one month later;

FIG. 4 is a picture showing cells alive that are tested by H & E staining after embedding 4×10⁶ parathyroid live cells into TheraCyte® three months later;

FIG. 5 is a picture showing parathyroid cells alive that are tested by immune tissue staining after embedding 4×10⁶ parathyroid cells into TheraCyte® three months later;

FIG. 6 is a picture showing cells alive that are tested by H & E staining after embedding 4×10⁵ parathyroid live cells into TheraCyte® three months later; and

FIG. 7 is a picture showing parathyroid cells alive that are tested by immune tissue staining after embedding 4×10⁵ parathyroid cells into TheraCyte® three months later.

### DETAILED DESCRIPTION OF THE PREFERRED EMBODIMENT

A therapeutic microencapsulation in accordance with the present invention is made by embedding at least 4×10⁵ parathyroid live cells in TheraCyte® (20 μl), wherein the parathyroid live cells are contained in −179° C. liquid nitrogen and then unfrozen for preparing the therapeutic microencapsulation.

Additionally, another preferred embodiment of the therapeutic microencapsulation in accordance with the present invention is made by embedding 4×10⁶ parathyroid live cells in TheraCyte® (20 μl), wherein the parathyroid live cells are contained in −179° C. liquid nitrogen and then unfrozen for preparing the therapeutic microencapsulation.

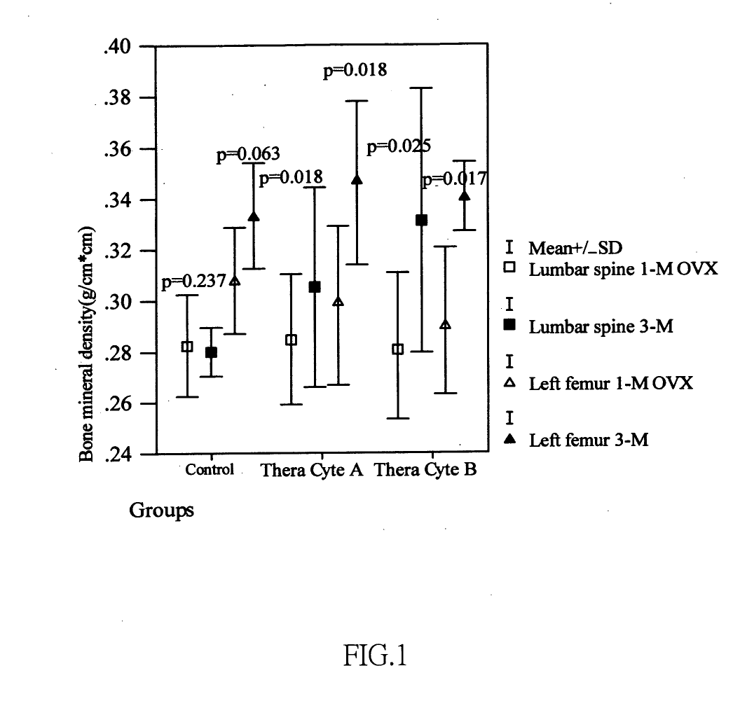

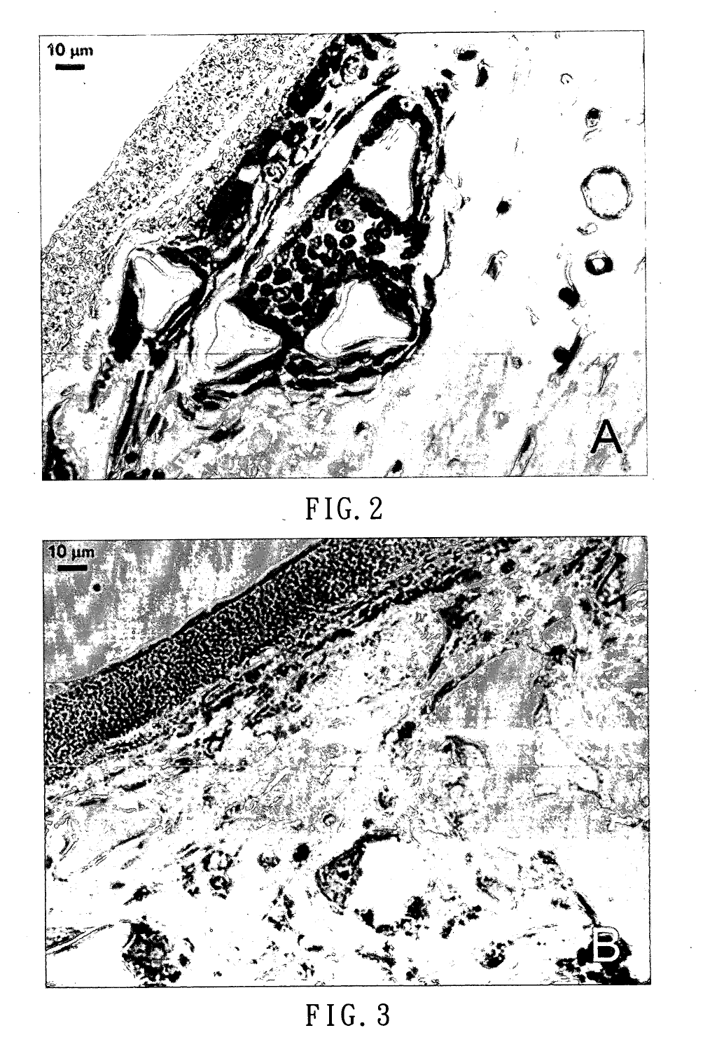

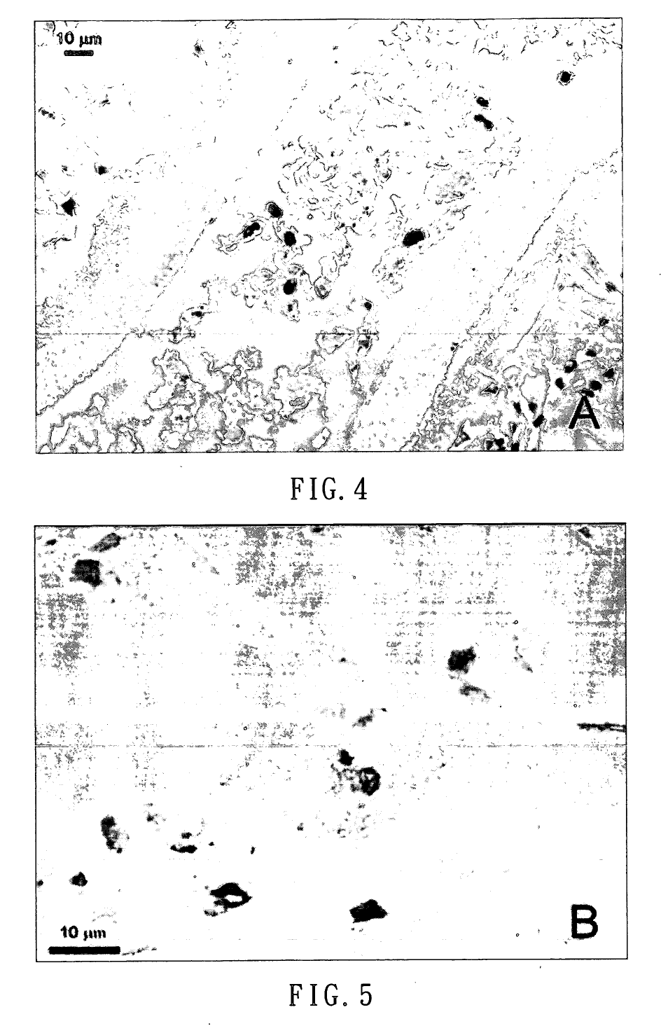

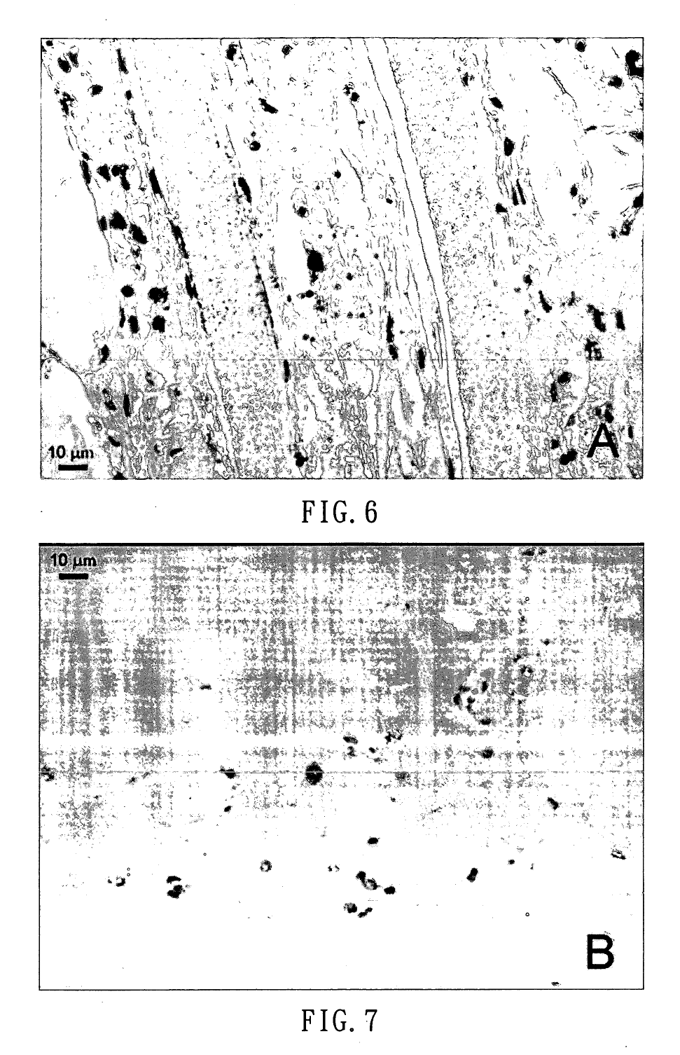

TheraCyte® is an encapsulation device purchasable from TheraCyte, Inc. 17511 Armstrong Ave, Irvine, Calif. 92614. As shown in FIGS. 1 to 7, an A-group (TheraCyte® A mentioned in the following tables) represented that each TheraCyte® (20 μl) unit was embedded with 4×10⁶ parathyroid live cells and had 7 TheraCyte® units implanted into subcutaneous tissue at backs of experimental white mice, wherein the white mice were ovariectomized one month ago. A B-group (TheraCyte® B mentioned in the following tables) represented that each TheraCyte® (4.5 μl) unit was embedded with 4×10⁵ parathyroid live cells and had 7 TheraCyte® units implanted into subcutaneous tissue at backs of ovariectomized white mice. Then, 7 white mice were simply cut subcutaneous tissues open to serve as a control group (Blank experiment).

All white mice had blood tests to check calcium, phosphorus and parathyroid hormone contents when the TheraCyte® (20 μl) units were implanted and then had the blood tests three times a month. All white mice were weighted just after ovariectomy and then weighted again four months later. Bone density at the lumbar spine (L1-L5) and the left femur of the each white mouse was measured by Dual-Enery-X-Ray Absorptiometry (DEXA) respectively after one-month period since ovariectomy and after three-month period since TheraCyte® unit implantation (i.e. four-month period after ovariectomy).

Survival rate of tissue cells after unfreezing is about 55 to 79%. All white mice gain more weight (P<0.001) after four months since ovariectomy and the weight gaining is meaningful to each group and has no difference between the groups. In the control group, the bone density of L1 to L2 is meaninglessly decreased (P=0.237) and the bone density of the left femur is meaninglessly increased (P=0.063).

After the three-month period since implantation of TheraCyte® unit, the bone density of the L1 to L5 in the A-group is meaningfully increased (P=0.018) and the bone density of the left femur in the A-group is meaningfully increased (P=0.018). After the three-month period since implantation of TheraCyte® unit, the bone density of the L1 to L5 in the B-group is meaningfully increased (P=0.025) and the bone density of the left femur is meaningfully increased (P=0.017). The parathyroid hormone content in the A-group is higher than the one in the B-group (P=0.002) and the parathyroid hormone content in the B-group is higher than the one in the control group (P=0.013). The blood calcium content in the A-group is higher than the one in the B-group (P=0.039) but the phosphorus content of blood in the A-group is the same with the one in the B-group (P=0.336).

The conclusion of above experiment is that the TheraCyte® unit having 4×10⁵ parathyroid live cells increases the bone density of the ovariectomized white mice within three months and the TheraCyte® unit having 4×10⁶ parathyroid live cells also has the same efficiency. However, the former unit can eliminate side effect of blood calcium increase.

Therefore, it is discovered that transplanting parathyroid live cells from hyperparathyroidism patients to hypoparathyroidism patients makes the hypoparathyroidism patients free from anti-immune treatment. Moreover, the unfrozen parathyroid cells enable to last lively about four months after the parathyroid gland is placed into −179° C. liquid nitrogen.

Embedding 4×10⁵ parathyroid live cells in the TheraCyte® unit enables to treat osteoporosis to ovariectomized white mice. Correspondingly, the therapeutic microencapsulation having 200 times of 4×10⁵ parathyroid live cells enables to treat osteoporosis in human body.

Embedding 4×10⁶ parathyroid live cells in the TheraCyte® unit enables to incur high blood calcium to ovariectomized white mice. Correspondingly, the therapeutic microencapsulation having 200 times of 4×10⁵ parathyroid live cells enables to apply to the hypoparathyroidism patient.

Results of the above experiment are shown in the following tables:

TABLE 1
Serum levels of iPTH (pg/ml) at 0-, 1-, 2-, and 3-months
after TheraCyte ® unit implantation are
shown. Using repeated measures of analysis of variance.
All data = mean ± SD.

| | Ovariectomy | 1-month | 2-months | 3-months |
|---|---|---|---|---|
| Control^{⋆#} | 4.74 ± 0.62 | 5.51 ± 0.98 | 4.67 ± 2.00 | 3.20 ± 2.29 |
| (N = 7) | | | | |
| TheraCyte | 5.43 ± 0.64 | 16.54 ± 0.90 | 15.26 ± 4.24 | 10.45 ± 1.43 |
| A^{∘#} | | | | |
| (N = 7) | | | | |
| TheraCyte | 5.94 ± 1.36 | 11.13 ± 0.44 | 7.29 ± 1.37 | 6.85 ± 1.14 |
| B^{⋆∘} | | | | |
| (N = 7) | | | | |

TheraCyte ® A: implantation of 4 × 10⁶ parathyroid live cells
TheraCyte ® B: implantation of 4 × 10⁵ parathyroid live cells
^{⋆}p = 0.013,
^{∘}p < 0.001,
^{#}p = 0.040

According to variations of parathyroid hormone in table 1, the TheraCyte® A implantation and the TheraCyte® B implantation both generate parathyroid hormone.

TABLE 2
Serum levels of calcium (mg/dl) at 0-, 1-, 2-, and 3-months after
TheraCyte ® implantation are shown by using repeated measures
of analysis of variance. All data = mean ± SD.

| | Oophorectomy | 1-month | 2-months | 3-months |
|---|---|---|---|---|
| Control^{#∘} | 10.4 ± 0.99 | 10.2 ± 0.55 | 9.5 ± 1.23 | 8.62 ± 1.37 |
| (N = 7) | | | | |
| TheraCyte | 9.2 ± 1.05 | 10.4 ± 0.34 | 10.4 ± 0.64 | 10.0 ± 2.11 |
| A^{⋆∘} | | | | |
| (N = 7) | | | | |
| TheraCyte | 9.4 ± 0.83 | 9.7 ± 0.24 | 9.8 ± 0.97 | 7.14 ± 1.62 |
| B^{⋆#} | | | | |
| (N = 7) | | | | |

TheraCyte ® A: implantation of 4 × 10⁶ parathyroid live cells
TheraCyte ® B: implantation of 4 × 10⁵ parathyroid live cells
^{⋆}p = 0.116,
^{∘}p > 0.999,
p = 0.476

According to data of calcium variation in table 2, the TheraCyte® A implantation and the TheraCyte® B implantation has no influence on calcium variation.

TABLE 3
Serum levels of phosphorus (mg/dl) at 0-, 1-, 2-, and 3-months after
TheraCyte ® implantation are shown by using repeated measures
of analysis of variance. All data = mean ± SD.

| | Ovariectomy | 1-month | 2-months | 3-months |
|---|---|---|---|---|
| Control^{∘#} | 5.8 ± 0.58 | 5.4 ± 0.36 | 4.4 ± 0.67 | 4.3 ± 0.52 |
| (N = 7) | | | | |
| TheraCyte A^{⋆∘} | 5.1 ± 0.53 | 5.1 ± 0.83 | 5.4 ± 0.42 | 6.2 ± 0.94 |
| (N = 7) | | | | |
| TheraCyte B^{⋆#} | 5.6 ± 1.1 | 4.8 ± 0.57 | 5.9 ± 0.82 | 5.6 ± 1.13 |
| (N = 7) | | | | |

TheraCyte ® A: implantation of 4 × 10⁶ parathyroid live cells
TheraCyte ® B: implantation of 4 × 10⁵ parathyroid live cells
^{⋆}p > 0.999,
^{∘}p = 0.143,
p = 0.078

Table 3 shows the variations of serum levels of phosphorus.

A manufacturing method for the therapeutic microencapsulation in accordance the present invention comprises the following steps.

Step 1: Obtaining a Parathyroid Gland

The parathyroid gland is obtained by separating a heaviest parathyroid gland in surgery from a mammal having repeating hyperparathyroidism.

Step 2: Refrigerating the Parathyroid Gland

The parathyroid gland is immerged by a composition composed of Roswell Park Memorial Institute solution (85%), dimethyl sulfoxide (10%) and fetal calf serum (5%) and then immerged in −179° C. liquid nitrogen. Wherein, the fetal calf serum is purchasable from Sigma-Aldrich Coporation. Box 145078 St. Louis, Mo. 63178.

Step 3: Isolating the Parathyroid Cells

The refrigerated parathyroid gland is unfrozen and sliced into piece to isolate the parathyroid cells, microspheres are suspended in a full-growth solution. The amount of the parathyroid live cells is calculated by an excluding method of trypan blue and then a trypsin/EDTA 0.05 solution is used to separate individualize the parathyroid live cells. Wherein, trypsin is purchasable from Gibco Invitrogen Corporation, Grand Island, N.Y. 12072 and subjected to turn parathyroid tissue into parathyroid cells.

Step 4: Embedding to Achieve the Therapeutic Microencapsulation

Lastly, 4×10⁵, 4×10⁶, 200×4×10⁵ and 200×4×10⁶ parathyroid live cells are embedded in Theracyte® to achieve the various therapeutic microencapsulations.

In characteristics, the sliced parathyroid gland after unfreezing is treated to isolate parathyroid cells by using collagenase II, i.e. Collagenase II® from Gibco Invitrogen Corporation, Grand Island, N.Y. 14072.

According to above description, the present invention is to embed at least 4×10⁵ parathyroid live cells in Theracyte® to achieve the therapeutic microencapsulations, wherein the parathyroid live cells is refrigerated in −179° C. liquid nitrogen before embedding. By refrigerating, the parathyroid live cells enable to last alive for four months. Therefore, the therapeutic microencapsulations are efficient to treat osteoporosis and hypoparathyroidism for about four months by implantation to avoid daily subcutaneous injection.

Although this invention has been described in its preferred form with a certain degree of particularity, it is understood that the present invention of the preferred form has been made only by way of example and that numerous changes in the details of construction and the combination and arrangement of parts any be resorted to without departing from the spirit and scope of the invention.

What is claimed is:

1. A therapeutic microencapsulation is made by embedding at least 4×10⁵ parathyroid live cells in TheraCyte®, wherein the parathyroid live cells are contained in −179° C. liquid nitrogen and then unfrozen for preparing the therapeutic microencapsulation.

2. The therapeutic microencapsulation as claimed in claim 1, wherein the therapeutic microencapsulation contains 4×10⁶ parathyroid live cells.

3. The therapeutic microencapsulation as claimed in claim 1, wherein the therapeutic microencapsulation contains at least 200×4×10⁵ parathyroid live cells for treating humans.

4. The therapeutic microencapsulation as claimed in claim 1, wherein the therapeutic microencapsulation contains 200×4×10⁶ parathyroid live cells for treating humans.

5. A manufacturing method for therapeutic microencapsulation comprising steps of:
obtaining a parathyroid gland, wherein the parathyroid gland is obtained by separating a heaviest parathyroid gland in surgery from a mammal having repeating hyperparathyroidism;
refrigerating the parathyroid gland, wherein the parathyroid gland is immerged by a composition composed of Roswell Park Memorial Institute solution (85%), dimethyl sulfoxide (10%) and fetal calf serum (5%) and contained in −179° C. liquid nitrogen;
isolating the parathyroid cells, wherein the refrigerated parathyroid gland is refrozen and sliced into piece to isolate the parathyroid cells, microspheres are suspended in a full-growth solution and then a trypsin/EDTA 0.05 solution is used to separate individualize parathyroid live cells; and
embedding to achieve the therapeutic microencapsulation, wherein 4×10⁵ parathyroid live cells are embedded in Theracyte® to achieve the therapeutic microencapsulation.

6. The manufacturing method as claimed in claim 5, wherein the sliced parathyroid gland after unfreezing is treated with collagenase II to isolate parathyroid cells.

7. The manufacturing method as claimed in claim 5, wherein an amount of the parathyroid live cells is calculated by an excluding method of trypan blue after separating the parathyroid cells with a trypsin/EDTA 0.05% solution.
